(19) United States
(12) Patent Application Publication
(10) Pub. No.: US 2008/0213815 A1
(43) Pub. Date: Sep. 4, 2008

(54) DIAGNOSING BREAST CANCER BY SEPRASE LEVEL

(75) Inventors: Brendan Francis O'Connor, Dublin (IE); Pamela O'Brien, Dublin (IE); Yvonne Antoinette Birney, Dublin (IE)

Correspondence Address: BROWN RUDNICK LLP, ONE FINANCIAL CENTER, BOSTON, MA, 02111, US

(73) Assignee: DUBLIN CITY UNIVERSITY, Dublin (IE)

(21) Appl. No.: 12/040,349

(22) Filed: Feb. 29, 2008

Related U.S. Application Data
(60) Provisional application No. 60/904,589, filed on Mar. 2, 2007.

(51) Int. Cl. C12Q 1/37

(52) U.S. Cl. 435/23

(57) ABSTRACT

A method of diagnosing subjects for breast cancer comprising the steps of (a) establishing the seprase level of a subjects test material and (b) determining those subjects with seprase levels above about 0.0008 nmoles.min⁻¹.mg⁻¹ (by wgt); or above about 0.05 nmoles.min⁻¹.ml⁻¹ (by volume) as positive for breast cancer and other epithelial cancers.

### CROSS-REFERENCE TO RELATED APPLICATION

This patent application claims priority to U.S. Provisional Application Ser. No. 60/904,589 filed in the U.S. Patent and Trademark Office on Mar. 2, 2007 by O'Brien et al., the entire contents of which being hereby incorporated by reference.

### BACKGROUND

1. Technical Field

The current disclosure relates to a method of diagnosing subjects for breast cancer and other epithelial cancers comprising the steps of (a) establishing the seprase level of a subjects test material and (b) determining those subjects with seprase levels above about 0.0008 nmoles.min⁻¹.mg⁻¹ (by wgt); or above about 0.05 nmoles.min⁻¹.ml⁻¹ (by volume) as positive for breast cancer and other epithelial cancers.

2. Background of the Invention

Annually, 9 million women worldwide undergo fine needle biopsy resulting in 1.2 million women being diagnosed with breast cancer. Breast cancer also occurs in men at about 1% of the rate of women. It is believed that earlier detection of breast cancer and earlier treatment yields better long-term prospects for subjects with the disease. It is important when patients are diagnosed with cancer, which doctors know whether the disease is local or has spread to other locations. It is this ability to spread to other tissues and organs that makes cancer a possibly life-threatening disease. As a result, there is great interest in understanding what makes metastasis possible for a cancerous tumour.

Without being bound by any particular theory, it is believed that metastasis is a complex series of steps in which cancer cells leave the original tumour site and migrate to other parts of the body via the bloodstream or lymphatic system. To do so, malignant cells break away from the primary tumour and attach to and degrade proteins that make up the surrounding extracellular matrix (ECM), which separates the tumour from adjoining tissue. By degrading these proteins, cancer cells are believed able to breach the ECM and escape.

Again, without being bound by any particular theory, it is believed that the invasion of tumour cells through surrounding tissue is an important stage of metastasis and requires the activity of certain types of enzymes. Tumour cell invasiveness has been linked with an increased production of extracellular matrix-degrading enzymes.

To date there is no validated serum or saliva tumour marker for breast cancer screening. There are three main methods of screening for breast cancer: mammography, clinical breast examination, and breast self-examination. A mammogram is a breast x-ray that is the most proven screening test for reducing the risk of dying from breast cancer. It is important to remember that breast cancer cannot be diagnosed by mammography alone. In addition, a patient may be asked to have further testing (e.g., ultrasound or biopsy) because something on the mammogram needs more evaluation. One study found that, while 11% of mammograms performed in the United States lead to additional evaluation; the lesion turns out to be benign in more than 90% of the time.

Tumour markers are substances occurring in blood, tissue or urine, that are associated with cancer and whose measurement or identification is useful in patient screening, diagnosis or clinical management. The markers, which are generally soluble molecules, are frequently glycoproteins. Glycoproteins are detectable by various methods including the use of monoclonal antibodies. The ideal tumour marker should be (1) specific for the cancer for which it is testing; (2) not present in any other conditions; and (3) of a concentration which changes with or reflects a characteristic of the cancer, such as the amount of malignant tissue present or its propensity to metastasize. An ideal tumour maker could be used for screening, diagnosis, monitoring of disease progression, or directing treatment options and be easily and reproducibly measured. Currently there are no ideal tumour markers and most are restricted to monitoring cancers once they have been detected and diagnosed using other methods.

There are no tumour makers which are currently recommended for screening of the general population. Most tumour markers have too many false positives from benign conditions to make screening useful. Many markers only clearly identify malignancy once the cancer is sufficiently advanced to make this detection of limited use.

Seprase activity is most often assessed by zymography, which is not a quantitative assay. A recent study established a relatively simple and quantitative method for determining seprase activity. The degradation of a 3H-gelatin substrate is measured in the presence of 5 mM EDTA which inhibits matrix metalloproteinases but not seprase. The quantitative character of the assay was demonstrated using partially purified seprase from chicken embryos, a preparation that lacks detectable matrix metalloproteinase activity. Additional experiments were performed to validate the quantification of seprase activity using the radiographic assay by comparing the results to zymography. Exposure to 22 or 37 degrees C. results in maximal seprase activity while exposure to 80 or 100 degrees C. completely abolishes seprase activity in both zymography and the radiographic assay. Exposure to 60 degrees C. abolished seprase activity as judged by zymography, but about 50% gelatinase activity was observed using the 3H-gelatin substrate.

Immunopreciptiation with a seprase-specific antibody specifically removed seprase and lowered the seprase activity remaining in the extracts as judged by both assays.

There is one recent report using the Prolyl Oligopeptidase (PO) substrate; Z-Gly-Pro-AMC (without any selective inhibitors) to detect recombinant seprase activity. This would not work in serum as PO and Seprase would both be naturally present and they both cleave the substrate, hence a specific PO inhibitor must be included (see summary of invention for explanation).

Seprase as a melanoma marker has been reported. The A recent article describes the identification of seprase activity in tissue utilising gelatin zymography, a non-quantitative assay. Seprase localisation at invadopodia was also visualised on tissue samples using confocal microscopy and an antibody to detect seprase. This method does not require the enzyme to be active to visualise the location of the enzyme. The findings of this paper relate to tissue and it does not discuss the detection of seprase activity in serum.

Another article recently investigated the inhibition of PO in primary neuronal cultures using a modification of our bovine assay. This modification allowed this group to investigate the true PO levels in sera of patients with bipolar disorder and schizophrenia. This group used the assay to determine another enzyme's levels in a separate disease from breast cancer.

Seprase is an enzyme that has the ability to degrade extracellular matrix components. Again, without being bound by any particular theory, it is believed that this process is essential to the cellular migration and matrix invasion that occurs during tumour invasion and metastasis. It is thought that seprase has a role in facilitating the tumour cell progression. Some report that the enzyme is over expressed by invasive tumour cells, a feature also observed in pathologic specimens of many epithelial cancers, including breast, lung, colon and pancreatic carcinomas. Until now there has been no early detection tumour marker test for breast cancer.

### SUMMARY

In one instance, the test is used during the initial assessment process of patients, in conjunction with the clinical investigation and mammogram/ultrasound. It is also valuable in situations where mammography cannot detect the breast cancer. By using this highly sensitive assay to detect this epithelial cancer serum marker, seprase, it will be possible to identify patients with early stages of breast cancer and other epithelial cancers and thereby increasing their chances of an improved prognosis.

The initial assessment of patients does not require an invasive procedure such as a biopsy. A minimal sample of blood (e.g. 1-2 ml serum) or saliva is all that is required to carry out the seprase biomarker test.

Seprase is also an important prognostic factor, indicating the possibility of metastasis, not only in breast cancer patients but also for the other epithelial cancers mentioned previously. The test could also be used to monitor a patient's response to treatment.

The substrate used for the test is Benzyloxycarbonyl (Z)-Gly-Pro-7-amino-4-methylcoumarin (AMC) [Z-Gly-Pro-AMC]. Seprase showed markedly less activity towards the substrate Ala-Pro-7-amino-4-trifluoromethylcoumarin (AFC). This substrate is for a completely different enzyme, namely DPPII.

PO which cleaves the substrate Z-Gly-Pro-AMC is also present in biological samples. In order to differentiate between the two enzyme activities, the addition of a specific PO inhibitor as shown in Table 5 is added to the assay. This allows for the quantitative analysis of seprase in the biological samples.

In particular embodiments, this test is useful in a kit for breast cancer screening, diagnosis, and for monitoring the progression of the disease.

This invention comprises a method of diagnosing subjects for breast cancer comprising the steps of establishing the seprase level of a subjects test material and determining those subjects with seprase levels above about 0.0008 nmoles.min⁻¹.mg⁻¹ (by wgt); or above about 0.05 nmoles.min⁻¹.ml⁻¹ (by volume) as positive for breast cancer. In some embodiments the test material is blood or a blood fraction such as serum. In others the test material is saliva or tissue. A wide variety of samples is envisioned according to the present disclosure.

In a specific embodiment the basic method of seprase level determination includes the steps of pre-incubating an aliquot of test fluid for 15 minutes at 37° C. with a titred concentration of specific PO inhibitor in a microtitre plate; adding 100 μM Z-Gly-Pro-AMC substrate; terminating reactions; and fluorimetrically determining the seprase level by weight or by volume.

### BRIEF DESCRIPTION OF THE DRAWINGS

FIG. 1 is a schematic of seprase activity.

FIG. 2 shows Total Activity of Serum Samples from full cohort of patients.

FIG. 3 shows Specific Activity of Serum Samples from full cohort of patients.

FIG. 4 is an AMC Standard Curve.

FIG. 5 is a Serum Quenched AMC Standard Curve.

### DETAILED DESCRIPTION OF THE INVENTION

This invention will be better understood with reference to the following definitions:

“Screening” as used herein shall mean that the assay is about 89% or greater sensitive and has a specificity of about 70% or greater. These results indicate that the assay detects about 89% of the positive samples as positive, (i.e. as having breast cancer) and about 70% of the samples correctly as negative (i.e. as not having breast cancer).

These results show that the current biomarkers are not as sensitive or specific as that of the early seprase biomarker. Many findings have also shown that a combination assay of tumour markers enhances the sensitivity and specificity.B. Diagnosis of breast cancer as used herein shall mean subjects or patients with seprase levels above about 0.0008 units/mg=0.0008 nmoles.min⁻¹.mg⁻¹ (by wgt); or above about 0.05 units/ml=0.05 nmoles.min⁻¹.ml⁻¹ (by volume) shall be deemed positive for breast cancer.

“Seprase titre/unit” shall mean the amount of enzyme which releases 1 nanomole of AMC per minute at 37° C. (unit=nmoles.min⁻¹).

“Test material” shall mean blood, blood fraction, tissue or saliva.

In particular embodiments, the practice of the current disclosure comprises fluorescence spectrometry using 7-amino-4-methyl-coumarin (AMC), particularly in a quantitative microplate seprase activity measurement. In particular embodiments, measurements are made of blood, blood fraction such as serum, tissue or saliva.

“Enzyme units” shall mean nanomoles of AMC released per minute at 37° C.

“Seprase” shall mean an integral membrane serine peptidase (also termed fibroblast activation protein and antiplasmin cleaving enzyme) with gelatinase activity (EC 3.4.21.-B28). It appears to act as a proteolytically active 170 kDa dimer consisting of two 97 kDa subunits. Seprase belongs to the S9b peptidase family, clan SC.

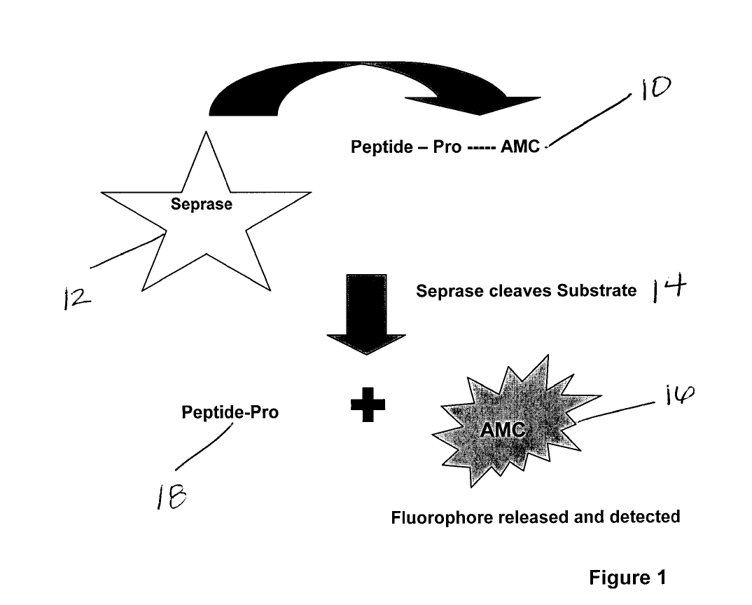

This disclosure generally relates to seprase enzyme activity as schematically shown in FIG. 1. The substrate is a peptide (with a proline as its final amino-acid) bound to the fluorophore AMC 10. Seprase 12 has an affinity for the proline-AMC bond in the substrate 10. Thus, the seprase 12 cleaves the proline-AMC bond shown as 14, detaching AMC 16 from the peptide 18. The resulting ‘free’ AMC 16 can subsequently be detected fluorimetrically.

The following examples describe the methods and procedures for diagnosing breast cancer in accordance with the present disclosure.

### EXAMPLE 1

### Seprase Determination

A 25 μl aliquot of serum was pre-incubated for 15 minutes at 37° C. with 5 μl of 2.5×10⁻⁴M JTP-4819 in 10% v/v MeOH in a 96 well microtitre plate prior to substrate addition. Next, 100 μl of 100μM Z-Gly-Pro-AMC substrate in 4% MeOH was added to the sample and the microtitre plate was incubated at 37° C. for 60 mins. Reactions were terminated by the addition of 170 μl of 1.5M acetic acid. Blanks or negative controls were prepared by adding 170 μl of 1.5M acetic acid to 25 μl of enzyme sample prior to substrate addition and incubation at 37° C. for 60 mins. The assay is performed in triplicate and at three different times. In biological samples (i.e. serum, saliva and tissue), it is often necessary to distinguish seprase activity from PO activity hence the addition of a specific PO inhibitor to the assay. PO was found to be sensitive to JTP-4819 at a concentration of 4×10⁻⁶M. This concentration is obtained by adding 5 μl of 2.5×10⁻⁴ M JTP-4819 to the assay.

Fluorimetric analysis of these samples was achieved using a Perkin Elmer LS50 Fluorescence Spectrophotometer at excitation and emission wavelengths of 370 nm and 440 nm respectively. Excitation slit widths were maintained at 10 nm while emission slit widths were adjusted accordingly for the range being analysed. Additional relative patient information was collected and is shown in Table 1.

End point measurements were allowed, as the enzyme assay was linear with respect to time and enzyme concentration up to 60 mins. Fluorimetric intensities observed were converted to nanomoles AMC released per minute per ml using the appropriate standard curve. Enzyme units were defined as nanomoles of AMC released per minute at 37° C.

TABLE 1
Characteristics of the patient cohort supplying serum samples.

| | | | | | | | | | Staging | | |
|---|---|---|---|---|---|---|---|---|---|---|---|
| | | | | Size | Node | | | Tumour | Liver US/ | | Follow |
| No. | Age | Sex | Diagnosis | (cm) | status | ER/PR | Grade | Markers | Bone Scan | Surgery | Up |
| 11 | 59 | F | IDC, ILC | 0.9 | 0/1 | +/+ | 2 | CEA = 1 | No M.D. | W.G WLE | N.A. |
| | | | | | (−) | | | | | and sn | |
| | | | | | | | | | | biopsy | |
| 12 | 61 | F | IDC | 1.9 | 0/1 | +/+ | 2 | CEA = 1 | No M.D. | W.G WLE | N.A. |
| | | | | | (−) | | | CA 15.3 = 10.4 | | and sn | |
| | | | | | | | | | | biopsy | |
| 13 | 58 | F | IDC | 1.1 | 2/2 | +/+ | 1 | N.D | No M.D. | W.G WLE | Further |
| | | | | | (+) | | | | | and sn | Axillary |
| | | | | | | | | | | biopsy | Clearance |
| 14 | 61 | F | IDC | 0.8 | 0/1 | +/+ | 2 | N.D | N.D. | W.G WLE | N.A. |
| | | | | | − | | | | | and sn | |
| | | | | | | | | | | biopsy | |
| 15 | 54 | F | IDC | 4 | 10/16 | +/+ | 3 | CEA = 3.2 | Liver ok. | Mastectomy | N.A. |
| | | | | | (+) | | | | Metastatic | and | |
| | | | | | | | | | deposit left | axillary | |
| | | | | | | | | | femur | clearance | |
| 16 | 49 | F | IDC | 2.5 | 6/23 | N.A. | 1 | CEA = 1.0 | No M.D. | W.G WLE | Further |
| | | | | | (+) | | | | | and sn | mastectomy |
| | | | | | | | | | | biopsy | and |
| | | | | | | | | | | | axillary |
| | | | | | | | | | | | clearance |
| 17 | 58 | F | IDC | 1.1 | (−) | +/+ | 2 | N.D | No M.D. | Mastectomy | N.A. |
| | | | | | | | | | | and | |
| | | | | | | | | | | axillary | |
| | | | | | | | | | | clearance | |
| 18 | 52 | F | IDC | 7 | 0/10 | +/+ | 2 | N.D. | No M.D. | Mastectomy | Reconstruction |
| | | | | | (−) | | | | | and | |
| | | | | | | | | | | axillary | |
| | | | | | | | | | | clearance | |
| 19 | 65 | F | IDC | 0.7 | 0/1 | +/+ | 1 | N.D. | No M.D. | W.G WLE | N.A. |
| | | | | | (−) | | | | | and sn | |
| | | | | | | | | | | biopsy | |
| 20 | N.A. | F | N.A. | N.A. | N.A. | N.A. | N.A. | N.A. | N.A. | N.A. | N.A. |

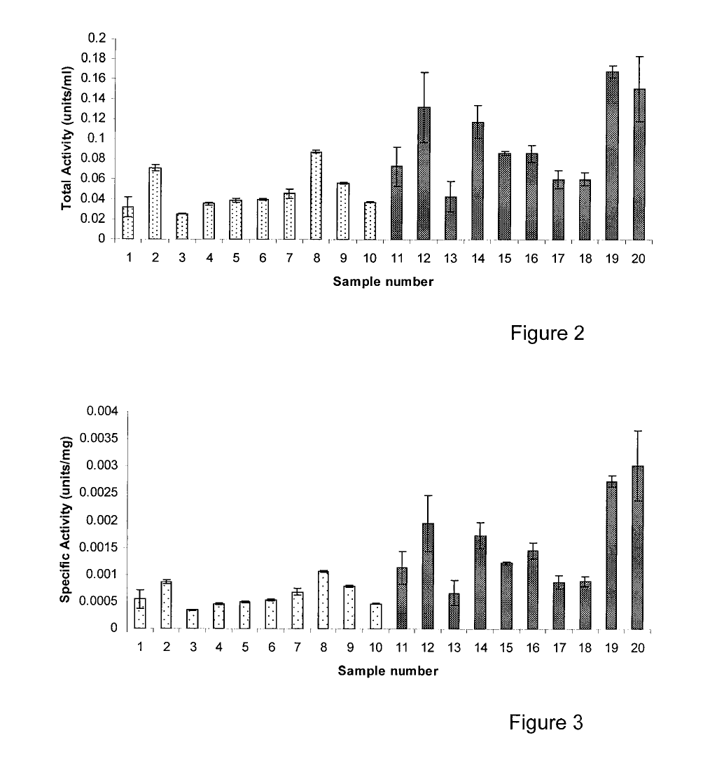

Table 1 shows the characteristics of the patient cohort supplying serum samples. Sample Numbers are listed as they appear in FIG. 2 and FIG. 3. The following abbreviations are used: N.D: not determined; N.A: not available; IDC: infiltrating ductal carcinoma; ILC: infiltrating lobular carcinoma; CEA: normal level 0-5; CA15.3: normal level 0-40; Node status: number of nodes positive for tumour cells/number of nodes examined; ER: estrogen receptor; PR: progesterone receptor; No M.D.: No metastatic disease; WG WLE; Wire guided WLE.

FIG. 2 shows Total Activity of Serum Samples from full cohort of patients. The control samples are numbered 1-10. Those patients with confirmed cases of IDC are numbered 11-20.

FIG. 3 shows Specific Activity of Serum Samples from full cohort of patients. As in FIG. 2, the control samples are numbered 1-10. Those patients with confirmed cases of IDC are numbered 11-20 (see Table 1).

Statistical analysis of both population data sets show that is was normally distributed (see Table 2) and, therefore, the Student's t-test was chosen to determine significance. A summary of the statistical analysis of the clinical data is shown in Tables 2, 3 and 4. Analysis shows that there is a highly significant difference between the control and cancer patients for both Total Activity (p=0.005) and Specific Activity (p=0.004) of seprase in serum (see Table 4). Therefore, it can be concluded from this that seprase levels are elevated in cancer patients with invasive ductal carcinoma and that the specific seprase assay described herein was able to detect this difference.

TABLE 2
Test of Normality of Clinical Samples

| | | Kolmogorov- | | |
|---|---|---|---|---|
| | | Smirnov(a) | | |
| | Diagnosis | Statistic | df | Sig. |
| Total Activity | Cancer | 0.211 | 10 | 0.200 |
| | Control | 0.239 | 10 | 0.109 |
| Specific Activity | Cancer | 0.165 | 10 | 0.200 |
| | Control | 0.243 | 10 | 0.097 |

TABLE 3
Group Statistics of Clinical Samples

| | Diagnosis | N | Mean | Std. Deviation | Std. Error Mean |
|---|---|---|---|---|---|
| Total Activity | Cancer | 10 | 0.09752 | 0.042259 | 0.013364 |
| | Control | 10 | 0.04695 | 0.019200 | 0.006072 |
| Specific Activity | Cancer | 10 | 0.00157058 | 0.000796097 | 0.000251748 |
| | Control | 10 | 0.00062619 | 0.000221251 | 0.000069966 |

TABLE 4
Independence Samples Test

| | | t-test for Equality of Means | | | | | | |
|---|---|---|---|---|---|---|---|---|
| | | | | | | | 95% | |
| | | | | | | | Confidence | |
| | | | | | | | Interval of the | |
| | | | | Sig. | Mean | Std. error | Difference | |
| | | t | df | (2-tailed) | Difference | Mean | Lower | Upper |
| Total | Equal variances not | 3.445 | 12.564 | 0.005 | 0.05057 | 0.01468 | 0.0004 | 0.0015 |
| Activity | assumed | | | | | | | |
| Specific | Equal variances not | 3.614 | 10.382 | 0.004 | 0.00094 | 0.00026 | 0.0004 | 0.0015 |
| Activity | assumed | | | | | | | |

Table 5 below lists examples of specific PO inhibitors and their associated potencies. This table is not exhaustive. Essentially, the lower the Kᵢ value (inhibition constant), the more potent the inhibitor.

TABLE 5
Non-exhaustive Prolyl Oligopeptidase Specific Inhibitors

| | Potency |
|---|---|
| Mammalian Inhibitor | |
| Fmoc-Ala-Pro-CN | Kᵢ = 5 nM |
| Fmoc-Pro-Pro-CN | Kᵢ = 5 nM |
| JTP-4819 | IC₅₀ = 0.83 nM; Kᵢ = 0.045 nM |
| ONO-1603 | IC₅₀ = 57 nM |
| S-17092-1 | Kᵢ = 1 nM |
| SUAM-1221 | Kᵢ = 190 μM |
| Y-27924 | IC₅₀ = 0.95 μM |
| Z-Indolinyl-Prolinal | Kᵢ = 2.4 nM |
| Z-Pro-3-fluoropyrrolidine | Kᵢ = 0.8 nM |
| Z-Pro-Prolinal | IC₅₀ = 0.74 nM; Kᵢ = 14 nM |
| Bacterial Inhibitor | |
| Eurystatin A | IC₅₀ = 0.004 μg/mL |
| Eurystatin B | IC₅₀ = 0.002 μg/mL |
| Postatin | IC₅₀ = 0.03 μg/mL |
| Propeptin | Kᵢ = 0.7 μM |
| SNA-8073-B | Kᵢ = 8 μM |
| Staurosporine | Kᵢ = 0.7 μM |

### EXAMPLE 2

### AMC Standard Curves

A 100 μM stock AMC solution containing 4% v/v methanol was prepared in 100 mM potassium phosphate, pH 7.4. All lower AMC concentrations were obtained using 100 mM potassium phosphate, pH 7.4 containing 4% v/v methanol as diluent. Stock solution and standards were stored in the dark at 4 □C for up to one month. Standard curves in the range 0-5 μM and 0-20 μM AMC were prepared in triplicate by combining 25 μl of 100 mM potassium phosphate pH 7.4, 100 μl of appropriate AMC concentration and 170 μl of 1.5M acetic acid. Fluorimetric analysis of these samples was achieved using a Perkin Elmer LS50 Fluorescence Spectrophotometer at excitation and emission wavelengths of 370 nm and 440 nm respectively. Excitation slit widths were maintained at 10 nm while emission slit widths were adjusted accordingly for the range being analysed.

The inner filter or quenching effect of enzyme samples was determined by combining 25 μl of enzyme sample, 100 μl range of AMC concentration and finally 170 μl of 1.5M acetic acid. The samples were all assayed in triplicate as described previously. The filtering effect of crude serum, tissue and saliva samples were each assessed. Even though the peptidase, seprase is also named ZIP (Z-Pro-prolinal Insensitive Peptidase), there are a number of potent and specific inhibitors of prolyl oligopeptidase, which can be utilised to distinguish between these peptidases. Table 5 exhibits a non-exhaustive list of some typical PO inhibitors which could be used.

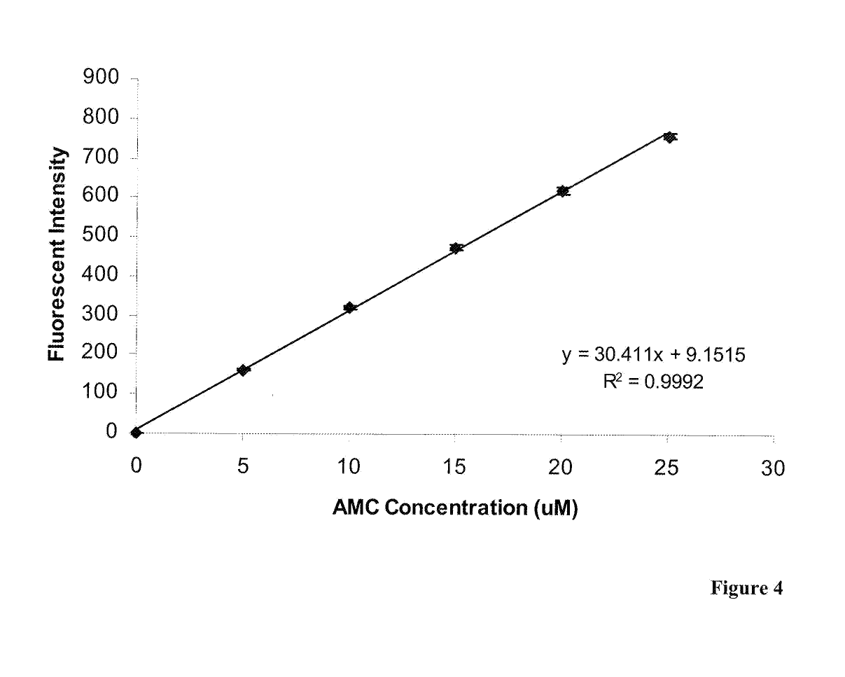

FIG. 4 shows a typical AMC standard curve. Plot of fluorescent intensity versus AMC concentration is presented. Excitation slit width was maintained at 10 nm, while the emission slit width was 2.5 nm.

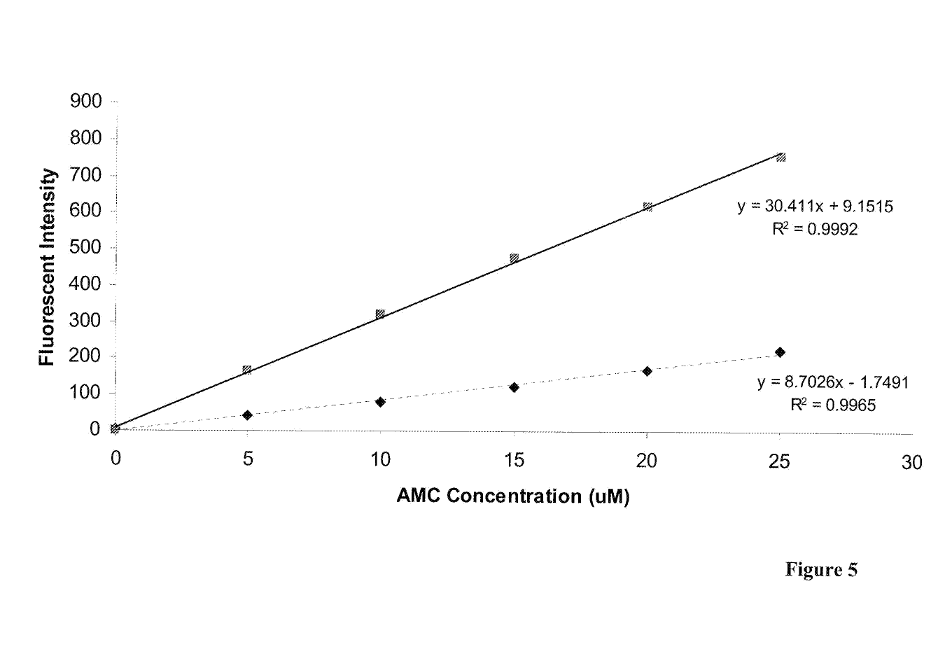

FIG. 5 shows an AMC Standard Curve incorporating the inner filter effect of serum (dotted line) by plotting fluorescent intensity versus AMC concentration.

Any references cited in this application are hereby incorporated by reference in their entirety for the substance of their disclosure. Those skilled in the art will recognize or be able to ascertain using no more than routine experimentation many equivalents of the specific embodiments of the invention described above. Such equivalents are intended to be encompassed herein.

What is claimed is:

1. A method of diagnosing subjects for breast cancer comprising the steps of:
establishing the seprase level of a subject's test material by
pre-incubating an aliquot of test fluid for 15 minutes at about 37° C. with 2.5×10⁻⁴M JTP-4819 in 10% v/v MeOH in a microtitre plate;
adding 100 μM Z-Gly-Pro-AMC substrate;
terminating reactions; and
fluorimetrically determining the seprase level by weight or by volume; and
determining which subjects were deemed positive for breast cancer.

2. The method of claim 1, wherein determining seprase levels above about 0.0008 units/mg=0.0008 nmoles.min⁻¹.mg⁻¹ (by weight) as positive for breast cancer.

3. The method of claim 1, wherein determining by seprase levels above about 0.05 nmoles.min⁻¹.ml⁻¹ (by volume) as positive for breast cancer.

4. The method of claim 2, wherein the test material is blood or a blood fraction.

5. The method of claim 3, wherein the test material is blood or a blood fraction.

6. The method of claim 2, wherein the test material is saliva.

7. The method of claim 3, wherein the test material is saliva.

8. The method of claim 2, wherein the test material is tissue.

9. The method of claim 3, wherein the test material is tissue.

10. A method of diagnosing subjects for breast cancer comprising the steps of:
establishing the seprase level of a subject's test material including the pre-incubation of an aliquot test fluid for a period of time;
fluorimetrically determining the seprase level by weight or by volume; and
determining which subjects were deemed positive for breast cancer.

11. The method of claim 10, wherein the step of establishing the seprase level of a subject's test material includes the steps of:
pre-incubating the aliquot of test fluid for 15 minutes at about 37° C. with 2.5×10−4M JTP-4819 in 10% v/v MeOH in a microtitre plate;
adding 100 μM Z-Gly-Pro-AMC substrate; and
terminating reactions.

12. The method of claim 10, wherein determining seprase levels above about 0.0008 units/mg=0.0008 nmoles.min⁻¹.mg⁻¹ (by weight) as positive for breast cancer.

13. The method of claim 1, wherein determining by seprase levels above about 0.05 nmoles.min⁻¹.ml⁻¹ (by volume) as positive for breast cancer.

14. A method of diagnosing subjects for breast cancer comprising the steps of:
establishing the seprase level of a subject's blood or blood fraction by pre-incubating an aliquot of test fluid;
adding Z-Gly-Pro-AMC substrate;
terminating reactions; and
fluorimetrically determining the seprase level by weight or by volume; and
determining which subjects were positive for breast cancer.

15. The method of claim 14, wherein the step of pre-incubating comprises pre-incubating an aliquot of test fluid for 15 minutes at about 37° C. with 2.5×10⁻4M JTP-4819 in 10% v/v MeOH in a microtitre plate.

16. The method of claim 14, wherein the amount of Z-Gly-Pro-AMC substrate added is 100 μM.
